(19) United States
(12) Patent Application Publication
(10) Pub. No.: US 2004/0010151 A1
(43) Pub. Date: Jan. 15, 2004

(54) Lansoprazole polymorphs and processes for preparation thereof

(76) Inventors: Nina Finkelstein, Herzliya (IL); Shomit Wizel, Petah Tiqva (IL)

Correspondence Address: KENYON & KENYON, ONE BROADWAY, NEW YORK, NY, 10004, US

(21) Appl. No.: 10/401,057

(22) Filed: Mar. 27, 2003

Related U.S. Application Data
(60) Provisional application No. 60/367,820, filed on Mar. 27, 2002.

(51) Int. Cl. C 07D 4 3/02

(52) U.S. Cl. 548/257

(57) ABSTRACT

The present invention relates to three crystalline solid forms of lansoprazole denominated as forms D, E and F. Processes for preparing these crystalline solid forms of lansoprazole are disclosed.

### CROSS-REFERENCE TO RELATED APPLICATION

This application claims the benefit under 35 U.S.C. § 1.119(e) of Provisional Application Serial No. 60/367,820 filed Mar. 27, 2002, the disclosure of which is incorporated by reference in its entirety herein.

### FIELD OF THE INVENTION

The present invention relates to lansoprazole crystalline solid forms and processes for their preparation.

### BACKGROUND OF THE INVENTION

Substituted 2-(2-pyridylmethyl) sulfinyl-1H-benzimidazole derivatives are well-known gastric proton pump inhibitors. These benzimidazole derivatives include lansoprazole, omeprazole, pantoprazole, and rabeprazole. They share the same function of inhibiting gastric acid secretion and thus are commonly used as anti-ulcer agents.

Lansoprazole represents one of the substituted benzimidazole derivatives and its chemical name is (2-[[[3-methyl-4-(2,2,2-trifluoro-ethoxy)-2-pyridinyl]methyl] sulfinyl]-1H-benzimidazole). The chemical structure of lansoprazole is:

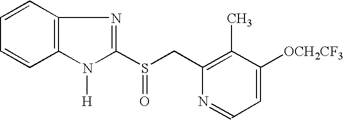

An amorphous form of lansoprazole prepared by spray drying method has been described (Farm. Vest. vol. 50, p. 347 (1999)).

Curin et al. describe an ethanole solvate form and an ethanole-hydrate form of lansoprazole (Farm. Vest. vol. 48, pp. 290-291 (1997).

Kotar et al. describe two lansoprazole polymorphs, designated as crystalline lansoprazole forms A and B, (Eur. J. Pharm. Sci. vol. 4, p. 182 (1996 Supp)). According to Kotar, each of the crystalline lansoprazole forms A and B exhibits a different DSC curve. In fact, crystalline lansoprazole form B is unstable and can undergo a solid-solid transition to form crystalline lansoprazole form A. Kotar provides no XRD data for crystalline lansoprazole forms A and B, and fails to disclose processes for preparing these crystalline forms.

Substituted 2-(2-pyridylmethylsulfinyl)-benzimidazole derivatives tend to lose stability and undergo decomposition when they contain traces of solvent in their crystal structure; this is so particularly when water is present in the crystals. Specifically, U.S. Pat. No. 6,002,011 and WO 98/21201 disclose solvent-free crystalline forms of lansoprazole. All of the cited references are incorporated by reference in their entireties.

The present invention relates to the solid state physical properties of lansoprazole. These properties can be influenced by controlling the conditions under which lansoprazole is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are determined by the conformation and orientation of molecules in the unit cell, which define a particular polymorphic form of a substance. A particular crystalline form may give rise to distinct spectroscopic properties that may be detectable by powder X-ray crystallography, or other parameters including solid state ¹³C NMR spectrometry and infrared spectrometry. The different physical properties permit one crystalline form to be distinguishable from another crystalline form as well as from that of the amorphous material.

No indication was found in the literature regarding the existence of other crystalline lansoprazole forms other than the known forms A, B, ethanolate and ethanolate-hydrate. There is a need to develop crystalline lansoprazole forms for better formulation.

### OBJECTS AND SUMMARY OF THE INVENTION

The present invention provides a crystalline solid form D of lansoprazole, characterized by an X-ray diffraction pattern having peaks at about 20.7, 23.8, 24.8, 25.2, 25.6 and 29.9±0.2 degrees two theta. Also, Form D may be characterized by a FTIR spectrum having absorption bands at 1168, 1186, 1440, 2975, 3301 and 3452 cm⁻¹. Form D may further be characterized by FTIR absorption bands 744, 825, 859, 917, 980, 1023, 1083, 1110, 1260, 1275, 1299, 1311, 1460, 1582, 2810, 2883 and 3014 cm⁻¹.

The present invention also provides a crystalline solid form E of lansoprazole, characterized by an X-ray diffraction pattern having peaks at about 18.5 and 19.8±0.2 degrees two theta. Form E may further be characterized by X-ray diffraction peaks at about 5.9, 9.0, 17.7 and 26.1±0.2 degrees two theta. Also, Form E may be characterized by a FTIR spectrum having absorption bands at 1168, 1186, 1440, 2975, 3301 and 3452 cm⁻¹. Form E may further be characterized by FTIR absorption bands at 744, 825, 859, 917, 980, 1023, 1083, 1110, 1260, 1275, 1299, 1311, 1460, 1582, 2810, 2883 and 3014 cm⁻¹.

The present invention also provides a crystalline solid form F of lansoprazole, characterized by an X-ray diffraction pattern having peaks at about 11.4, 14.4, 17.1, 22.9, 28.7 and 34.7±0.2 degrees two theta. Also, Form F may be characterized by a FTIR spectrum having absorption bands at 922, 1040, 1117, 1163, 1266, 1282, 1402, 1456, 2931, 2985 and 3235 cm⁻¹. Form F may further be characterized by FTIR absorption bands at 750, 801, 813, 857, 972, 1087, 1172, 1243, 1254, 1299, 1308, 1443, 1476 and 1581 cm⁻¹.

The present invention provides methods for preparing crystalline lansoprazole form A, comprising the steps of: a) preparing a solution of lansoprazole in a solvent selected from the group consisting of methanol, n-butanol, acetone, methylethylketone, ethyl acetate, dimethyl sulfoxide, dimethylformamide and their mixtures optionally with water; and b) isolating crystalline lansoprazole form A.

The lansoprazole in the preparing step includes amorphous and other crystalline solid forms of lansoprazole. Preferably, the lansoprazole in the preparing step is crystalline lansoprazole form A.

Optionally, the solvent may contain water. Preferably, the solvent containing water is selected from the group consisting of methanol, n-butanol, acetone, dimethyl sulfoxide and dimethylformamide. Preferably, the solvent is heated to a temperature higher than ambient temperature; more preferably, the temperature is the reflux temperature of the solvent. The reflux temperature for different solvents varies depending on the solvent, usually the temperature is between about 55 to about 80° C. The temperature range is dependent on stability and solubility of lansoprazole during heating.

The isolating step further comprises the steps of: c) precipitating the lansoprazole; and d) drying the lansoprazole to yield crystalline lansoprazole form A. Preferably, the precipitating step is performed by cooling the solution. Preferably, the solvent is cooled to ambient temperature.

The present invention provides a method of preparing crystalline solid lansoprazole form D, comprising the steps of: a) preparing a solution of lansoprazole in a solvent comprising 2-propanol and water; and b) isolating crystalline solid lansoprazole form D.

The lansoprazole in the preparing step includes amorphous and other crystalline solid forms of lansoprazole. Preferably, the lansoprazole in the preparing step is crystalline lansoprazole form A.

Preferably, the 2-propanol and water in the solution is present in a vol./vol. ratio of about 97.5/2.5; about 95/5; about 80/20; or about 60/40. Preferably, the isolating step is performed by filtering under vacuum.

Preferably, the solution is heated higher than the ambient temperature. More preferably, when the vol./vol. ratio of 2-propanol and water in the solution is 97.5/2.5 or 95/5, the solution is heated to reflux temperature; and when the vol./vol. ratio of 2-propanol and water in the solution is 80/20 or 60/40, the solution is heated to between about 55 to about 80° C.

The present invention provides a method of preparing crystalline solid lansoprazole form E, comprising the steps of: a) preparing a solution of lansoprazole in a solvent comprising 2-propanol and water; b) isolating the lansoprazole; and c) drying the isolated lansoprazole at a temperature below about 40° C. to yield crystalline solid lansoprazole form E.

The lansoprazole in the preparing step includes amorphous and other crystalline solid forms of lansoprazole. Preferably, the lansoprazole in the preparing step is crystalline lansoprazole form A. Preferably, the preparing step is performed by heating the solution to a temperature higher than ambient temperature. Preferably, the solution is heated to reflux temperature. Preferably, the lansoprazole in step (b) is the crystalline solid lansoprazole form E. Preferably, the isolating step further comprises the step of cooling the lansoprazole. Preferably the cooling step is performed by cooling the solution to ambient temperature.

Preferably, the drying step is performed under reduced pressure. Preferably, the drying step is performed at ambient temperature. More preferably, the drying step is performed overnight and at 20 mmHg.

The present invention provides a process for preparing crystalline solid lansoprazole form E, comprising the step of drying crystalline solid lansoprazole form D; preferably at ambient temperature, at reduced pressure (e.g., 20 mmHg) for a period of time (e.g., overnight)).

The present invention provides a method of preparing amorphous lansoprazole form, comprising the steps of: a) preparing a solution of lansoprazole in a solvent comprising 2-propanol and water; b) isolating the lansoprazole; and c) drying the isolated lansoprazole at a temperature between about 40° C. to 50° C. to yield amorphous lansoprazole form.

The lansoprazole in the preparing step includes amorphous and other crystalline solid forms of lansoprazole. Preferably, the lansoprazole in the preparing step is crystalline lansoprazole form A. Preferably, the preparing step is performed by heating the solution to a temperature higher than ambient temperature. Preferably, the solution is heated to reflux temperature.

Preferably, the isolated lansoprazole in step (b) is the crystalline solid lansoprazole form D. Preferably, the isolating step further comprises the step of cooling the lansoprazole. Preferably, the step of cooling is performed by cooling the solution to ambient temperature. More preferably, form D is converted to an amorphous form of lansoprazole comprising the step of drying crystalline lansoprazole form D; preferably between about 40 to about 50° C.

The present invention provides a method of preparing a mixture of crystalline solid lansoprazole form A and form D, comprising the steps of: a) dissolving or slurrying lansoprazole in a solvent comprising 2-propanol solvent; b) isolating mixture of crystalline solid lansoprazole form A and form D.

The lansoprazole in the preparing step includes amorphous and other crystalline solid forms of lansoprazole. Preferably, the lansoprazole in the step (a) is crystalline lansoprazole form A.

Preferably, the slurrying step is performed for about 70 hours. Preferably, the isolating step is performed by filtering under vacuum. Preferably, the product contains about 50% wt crystalline lansoprazole form A and 50% wt crystalline lansoprazole form D.

The present invention provides a method of preparing lansoprazole form E, comprising the step of grinding lansoprazole. Preferably the starting material is crystalline solid lansoprazole form D. Preferably the lansoprazole is ground by a mortar and a pestle.

The present invention provides a method of preparing lansoprazole form F, comprising the steps of: a) preparing a solution of lansoprazole in a solvent comprising methanol; b) exposing the solution to saturated methanol/water vapor; and c) isolating the crystalline solid lansoprazole form F.

The lansoprazole in the preparing step includes amorphous and other crystalline solid forms of lansoprazole. Preferably, the lansoprazole in the preparing step is crystalline lansoprazole form A.

Preferably, the exposing step is performed by keeping the solution in a closed system saturated with methanol and water vapor. Preferably, the exposing step is performed at about 25° C. for about two weeks.

The present invention provides crystalline solid lansoprazole forms D, E and F to be prepared by the processes disclosed above.

The present invention provides pharmaceutical compositions comprising an effective amount of at least one crystalline solid form of lansoprazole selected from the group consisting of crystalline solid lansoprazole forms D, E and F, and a pharmaceutical acceptable excipient.

### BRIEF DESCRIPTION OF THE DIAGRAMS

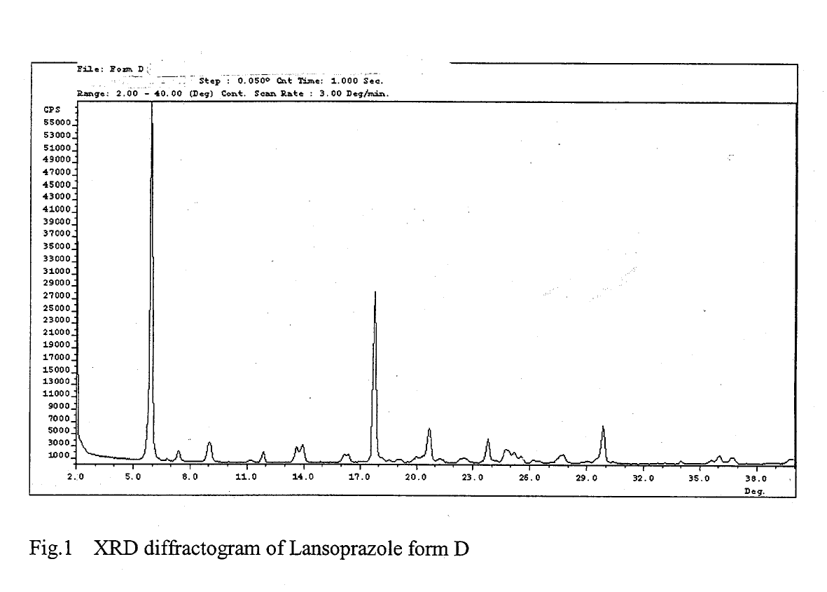

FIG. 1 represents the X-ray diffraction pattern of crystalline lansoprazole form D.

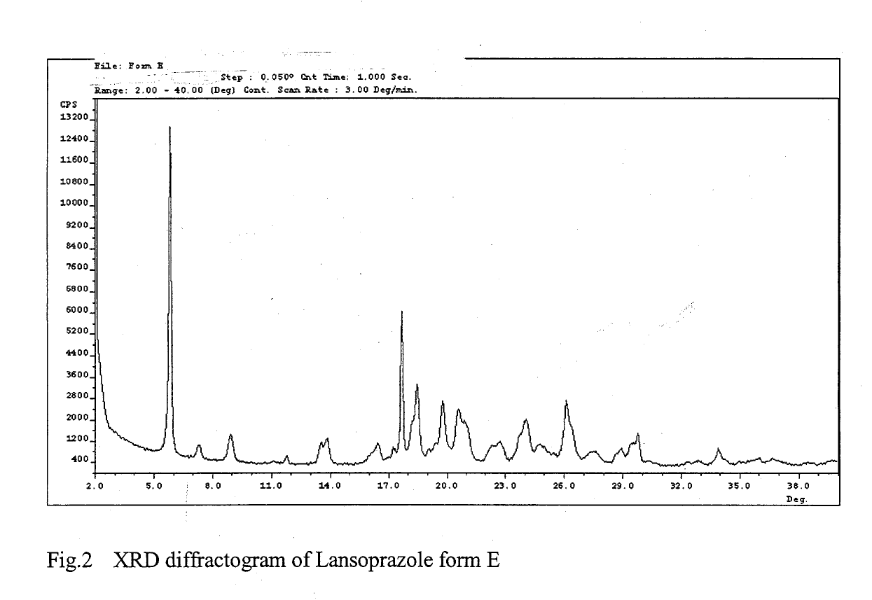

FIG. 2 represents the X-ray diffraction pattern of crystalline lansoprazole form E.

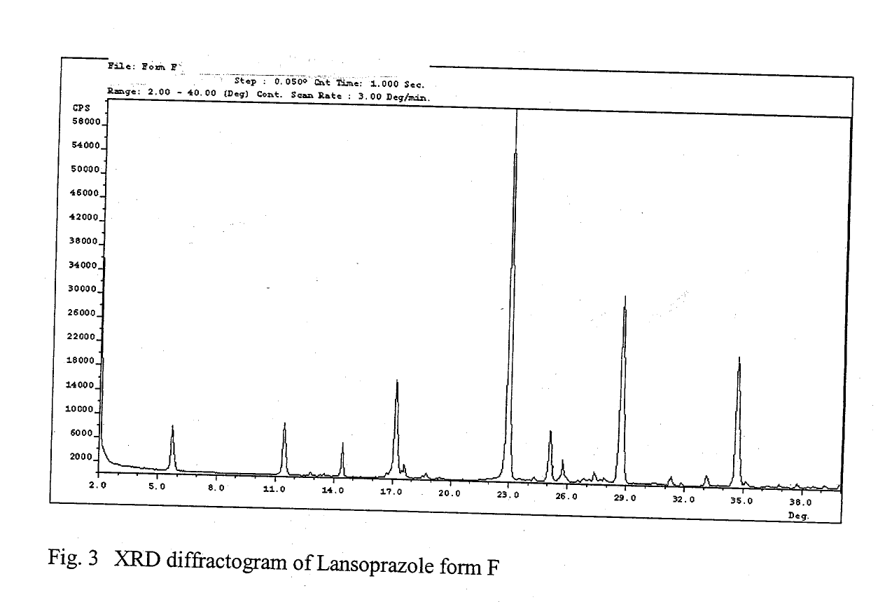

FIG. 3 represents the X-ray diffraction pattern of crystalline lansoprazole form F.

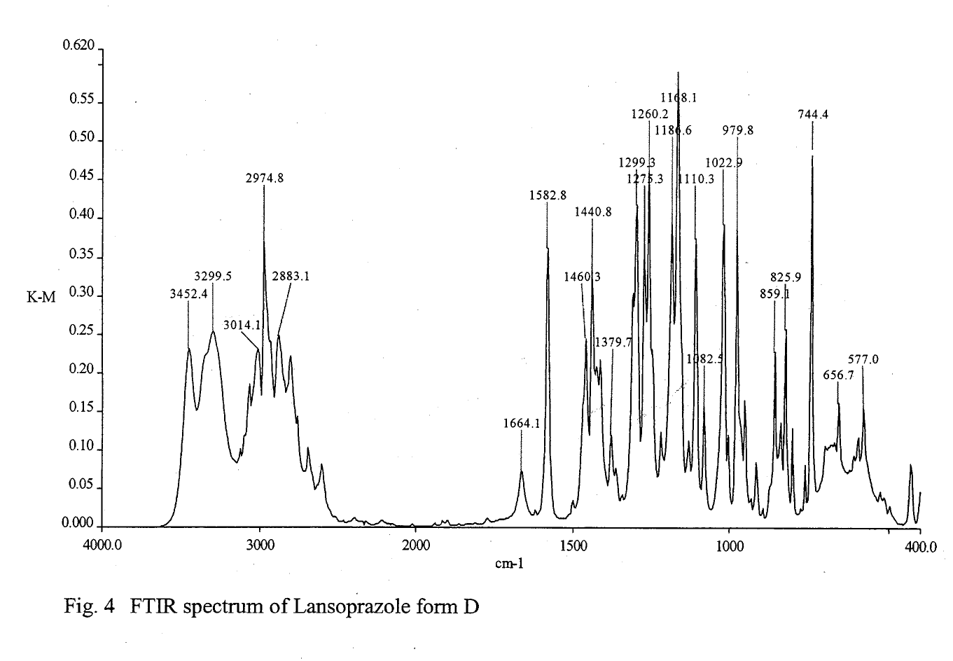

FIG. 4 represents the FTIR spectrum of crystalline lansoprazole form D.

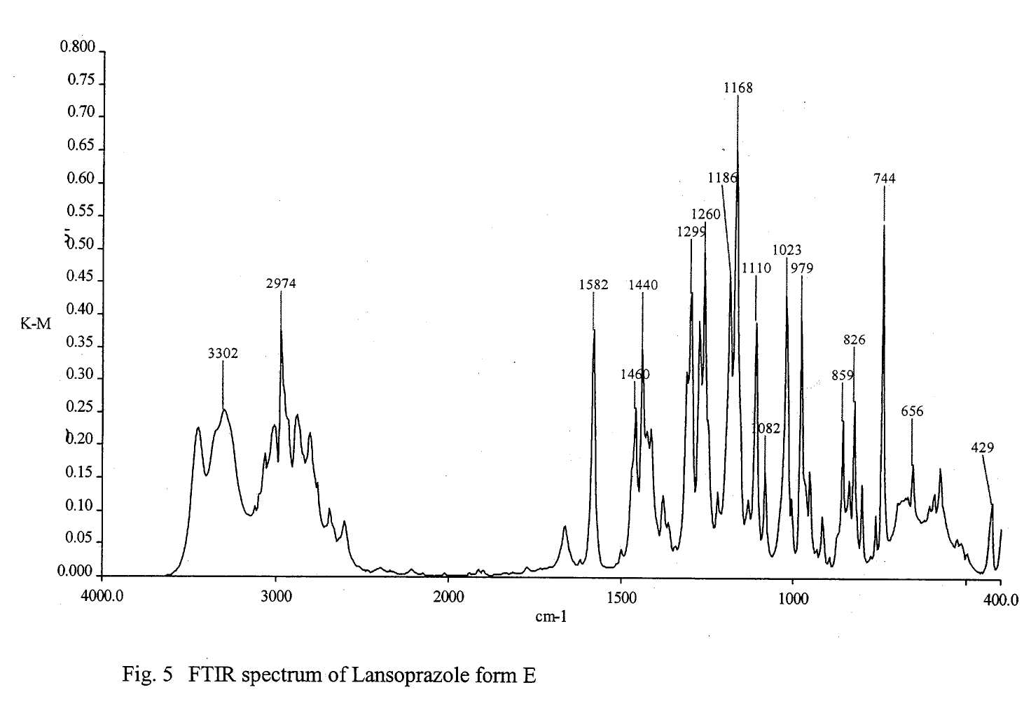

FIG. 5 represents the FTIR spectrum of crystalline lansoprazole form E.

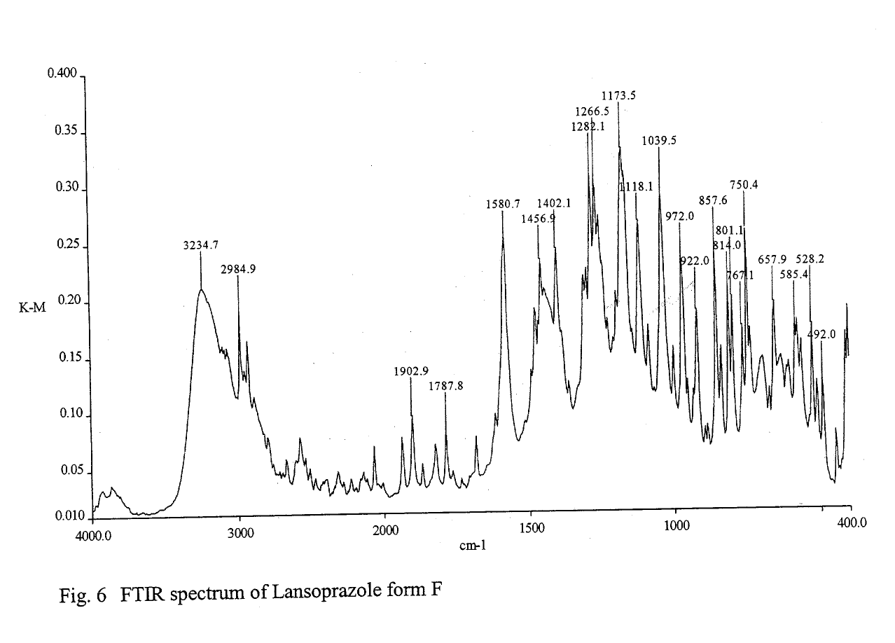

FIG. 6 represents the FTIR spectrum of crystalline lansoprazole form F.

### DETAILED DESCRIPTION OF THE INVENTION

Definitions:

As used herein, the following abbreviations are used: “DMSO” refers to dimethyl sulfoxide; “DMA” refers to dimethylamine; “DMF” refers to dimethylformamide; “FTIR” refers to Fourier Transform Technology, “grinding” refers to reducing a solid into fine particles; “slurrying” refers to forming a fluid suspension of particles having the consistency of cream.

Ambient temperature refers to a room temperature of about 20° C. to about 25° C.

The present invention relates to the crystalline forms of lansoprazole. Different crystal forms of lansoprazole may possess different physical properties including, for example, the flowability of the milled solid. Flowability affects the ease with which the material is handled during processing into lansoprazole. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important physical property of crystalline lansoprazole forms may relate to its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

The properties of these crystalline forms of lansoprazole may differ from that of crystalline lansoprazole forms A, B, ethanolate, ethanolate-hydrate and amorphous lansoprazole. They include solubility, stability, hygroscopicity (ability to remove moisture from air), tabletability, bioavailability, storage life (shelf life), and flow properties.

The three crystalline lansoprazole forms disclosed herein are prepared by the following methods:

i) crystalline lansoprazole forms A and D are formed by crystallization of crystalline lansoprazole form A from a solvent;

ii) crystalline lansoprazole form E is formed by drying crystalline lansoprazole form D;

iii) crystalline lansoprazole form F is formed by crystallization whereby the crystalline form of lansoprazole is induced to form by exposing a crystalline form of lansoprazole to methanol and water vapor; and

iv) crystalline lansoprazole form E is further formed by grinding lansoprazole.

Preferably, the lansoprazole is ground by a mortar and a pestle. Optionally, grinding includes mixing lansoprozole form D with a minimal amount of solvent (e.g., a mixture of 2-propanol and water) insufficient to dissolve lansoprazole form D. Preferably, the mixing is achieved by stirring the mixture at room temperature for the time needed to cause the desired transformation to yield crystalline lansoprazole form E. Preferably, the mixture is stirred for a period of 24 hours. Preferably, the resulting solid is filtered to separate crystalline lansoprazole form E.

X-Ray Powder Diffraction Patterns

All X-ray powder (XRD) diffraction patterns were obtained by methods known in the art. A Scintag X'TRA X-ray powder diffractometer, equipped with a solid state Si(Li) detector, thernoelectrically cooled, at a scanning speed of 30 min.⁻¹, scanning range of 2-40 degrees two-theta, copper radiation of 1.5418 was used.

FTIR Spectroscopy

All the FTIR spectra for the three crystalline forms of lansoprazole were collected on Perkin-Elmer spectrum One Spectrometer, using Diffuse Reflectance Technique. The solid-state FTIR spectra of many polymorphic systems often are found to be only slightly different, indicating that the pattern of molecular vibrations is not grossly affected by differences in crystal structure. (See, Drugs and the Pharmaceutical Sciences vol. 95, page 258, “Polymorphism in Pharmaceutical Solids” Edited by Harry G. Brittain, 1999).

According to one embodiment, the present invention provides crystalline lansoprazole form D, which is characterized by the following XRD peaks: 20.7, 23.8, 24.8, 25.2, 25.6 and 29.9±0.2 degrees two theta. A typical X-ray diffraction diagram of lansoprazole form D is shown in FIG. 1.

Crystalline lansoprazole form D produces a FTIR spectrum with characteristic absorption bands at about 1168, 1186, 1440, 2975, 3301 and 3452 cm⁻¹. Further FTIR bands were observed at about 744, 825, 859, 917, 980, 1023, 1083, 1110, 1260, 1275, 1299, 1311, 1460, 1582, 2810, 2883 and 3014 cm⁻¹. The FTIR spectrogram of lansoprazole form D is shown in FIG. 4.

According to one embodiment, the present invention provides crystalline lansoprazole form E, which is characterized by the following XRD peaks: 18.5 and 19.8±0.2 degrees two theta. Crystalline lansoprazole form E also exhibits X-ray reflections at 5.9, 9.0, 17.7 and 26.1±0.2 degrees two theta. A typical X-ray diffraction diagram of lansoprazole form E is shown in FIG. 2.

Crystalline lansoprazole form E produces a FTIR spectrum with characteristic absorption bands at about 1168, 1186, 1440, 2975, 3301 and 3452 cm⁻¹. Further FTIR bands were observed at about 744, 825, 859, 917, 980, 1023, 1083, 1110, 1260, 1275, 1299, 1311, 1460, 1582, 2810, 2883 and 3014 cm⁻¹. The FTIR spectrogram of lansoprazole form E is shown in FIG. 5.

According to one embodiment, the present invention provides crystalline lansoprazole form F, which is characterized by the following XRD peaks: 11.4, 14.4, 17.1, 22.9, 28.7 and 34.7±0.2 degrees two theta. A typical X-ray diffraction diagram of lansoprazole form F is shown in FIG. 3.

Crystalline lansoprazole form F produces a FTIR spectrum with characteristic absorption bands at about 922, 1040, 1117, 1163, 1266, 1282, 1402, 1456, 2931, 2985 and 3235 cm⁻¹. Further FTIR bands were observed at about 750, 801, 813, 857, 972, 1087, 1172, 1243, 1254, 1299, 1308, 1443, 1476 and 1581 cm⁻¹. The FTIR spectrogram of lansoprazole form F is shown in FIG. 6.

The invention will now be exemplified by the following non-limiting Examples.

### EXAMPLES

### Preparation of Lansoprazole Form A

Crystalline lansoprazole form A was obtained by re-crystallization of crystalline lansoprazole form A from solvents such as methanol, n-butanol, acetone, methylethylketone, ethyl acetate, DMSO or DMF. Crystallization solvents such as methanol, n-butanol, acetone, DMSO and DMF may contain water.

### Example 1

Crystalline lansoprazole form A (5.0 grams) was dissolved in methanol (30 mL). The methanol solution was heated to reflux. The methanol solution was then cooled to ambient temperature to induce precipitation of lansoprazole. The crystalline lansoprazole was filtered out from the methanol suspension under vacuum. The precipitate was dried at 40° C. under vacuum overnight to yield crystalline lansoprazole form A (yield: 2.7 grams).

### Preparation of Crystalline Lansoprazole Forms D and E

### Example 2

Crystalline lansoprazole form A (5.0 grams) was dissolved in a solution mixture (65 mL) containing 2-propanol and water (v/v=95:5). The solution mixture was heated at reflux to dissolution. The solution mixture was then cooled to ambient temperature to induce precipitation of lansoprazole. The lansoprazole precipitate was filtered out from the solution mixture under vacuum. Crystalline lansoprazole form D (wet precipitate sample) was obtained.

The wet precipitate sample was dried at ambient temperature under vacuum (20 mm Hg) overnight to yield crystalline lansoprazole form E (yield: 4.9 grams).

Drying of the wet precipitate sample at 40° C. gave the amorphous form of lansoprazole.

### Example 3

Crystalline lansoprazole form A (5.0 grams) was dissolved in 65 mL of a solution mixture of 2-propanol and water (v/v=97.5:2.5). The solution mixture was heated at reflux to dissolution. The solution mixture was then cooled to ambient temperature to induce precipitation of lansoprazole. The lansoprazole precipitate was filtered out from the solution mixture under vacuum. Crystalline lansoprazole form D (wet precipitate sample) was obtained.

The wet precipitate sample was dried at ambient temperature under vacuum (20 mm Hg) overnight to yield crystalline lansoprazole form E (yield: 4.9 grams).

Drying of the wet precipitate sample at 40° C. gave the amorphous form of lansoprazole.

### Example 4

Crystalline lansoprazole form A (5.0 grams) was dissolved in 50 mL of a solution mixture of 2-propanol and water (v/v=80:20). The solution mixture was heated to 80° C. to dissolution. The solution mixture was then cooled to ambient temperature to induce precipitation of lansoprazole. The lansoprazole precipitate was filtered out from the solution mixture under vacuum. Crystalline lansoprazole form D (wet precipitate sample) was obtained.

The wet precipitate sample was dried at ambient temperature under vacuum (20 mm Hg) overnight to yield crystalline lansoprazole form E (yield: 4.9 grams).

Drying of the wet precipitate sample at 40° C. gave the amorphous form of lansoprazole.

### Example 5

Crystalline lansoprazole form A (5.0 grams) was dissolved in (50 mL) of a solution mixture of 2-propanol and water (v/v=60:40). The solution mixture was heated at 80° C. to dissolution. The solution mixture was then cooled to ambient temperature to induce precipitation of lansoprazole. The lansoprazole precipitate was filtered out from the solution mixture under vacuum. Crystalline lansoprazole form D (wet precipitate sample) was obtained.

### Preparation of a Mixture of Crystalline Lansoprazole Form A and Form D

### Example 6

Crystalline lansoprazole form A (1.0 gram) was stirred in a solution mixture of 2-propanol and water (v/v=99.9:0.1) (10 mL) at ambient temperature for about 70 hours. The suspension was filtered under vacuum. The obtained wet precipitate product consisted of a mixture of crystalline lansoprazole forms A and D. The resulting mixture contained approximately 50% of each crystal form.

### Conversion of Lansorprazole Crystalline Form D to Form E

### Example 7

A wet sample of crystalline lansoprazole form D obtained in examples 2-5 was ground by a mortar and a pestle. The lansoprazole crystals obtained were designated to be crystalline lansoprazole form E.

### Preparation Crystalline Lansoprazole Form F

### Example 8

Crystalline lansoprazole form A (2 grams) was dissolved in 55 mL of methanol solution (methanol:water v/v=50:50). The methanol solution (14 mL) was put in a glass beaker, which was introduced into a bigger vessel (vessel volume of 125 mL), containing 14 mL of water. The vessel was kept closed at room temperature for two weeks. The resulting lansoprazole precipitate (wet) was designated to be crystalline lansoprazole form F.

Pharmaceutical Composition of Lansoprazole

In addition to the active ingredient(s), lansoprazole pharmaceutical compositions of the present invention may contain one or more excipients. Excipients are added to the composition for a variety of purposes.

Diluents increase the bulk of a solid pharmaceutical composition and may make a pharmaceutical dosage form containing the composition easier for the patient and care giver to handle. Diluents for solid compositions include, for example, microcrystalline cellulose (e.g. Aviccl7), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit7), potassium chloride, powdered cellulose, sodium chloride, sorbitol and talc.

Solid pharmaceutical compositions that are compacted into a dosage form like a tablet may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel7), hydroxypropyl methyl cellulose (e.g. Methocel7), liquid glucose, magnesium aluminum silicate, maltodextrin, methylcellulose, polymethacrylates, povidone (e.g. Kollidon7, Plasdone7), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol7, Primellose7), colloidal silicon dioxide, croscarmellose sodium, crospovidone (e.g. Kollidon7, Polyplasdone7), guar gum, magnesium aluminum silicate, methyl cellulose, microcrystalline cellulose, polacrilin potassium, powdered cellulose, pregelatinized starch, sodium alginate, sodium starch glycolate (e.g. Explotab7) and starch.

Glidants can be added to improve the flow properties of non-compacted solid compositions and improve the accuracy of dosing. Excipients that may function as glidants include colloidal silicon dixoide, magnesium trisilicate, powdered cellulose, starch, talc and tribasic calcium phosphate.

When a dosage form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level.

Selection of excipients and the amounts to use may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges as well as liquid syrups, suspensions and elixirs. An especially preferred dosage form of the present invention is a tablet.

A number of embodiments of the invention have been described. The present invention is not to be limited in scope by the specific embodiments described herein. It will be understood that various modifications may be made without departing from the spirit and scope of the invention.

What is claimed is:

1. A crystalline solid form of lansoprazole, characterized by data selected from the group consisting of an X-ray diffraction pattern having peaks at about 20.7, 23.8, 24.8, 25.2, 25.6 and 29.9±0.2 degrees two theta and a FTIR spectrum having absorption bands at 1168, 1186, 1440, 2975, 3301 and 3452 cm⁻¹.

2. The crystalline solid form of lansoprazole of claim 1, further characterized by an X-ray diffraction pattern substantially as depicted in FIG. 1.

3. The crystalline solid form of lansoprazole of claim 1, further characterized by FTIR absorption bands at 744, 825, 859, 917, 980, 1023, 1083, 1110, 1260, 1275, 1299, 1311, 1460, 1582, 2810, 2883 and 3014 cm⁻¹.

4. The crystalline solid form of lansoprazole of claim 1, further characterized by a FTIR spectrum substantially as depicted in FIG. 4.

5. A crystalline solid form of lansoprazole, characterized by data selected from the group consisting of an X-ray diffraction pattern having peaks at about 18.5 and 19.8±0.2 degrees two theta and a FTIR spectrum having absorption bands at 1168, 1186, 1440, 2975, 3301 and 3452 cm⁻¹.

6. The crystalline solid form of lansoprazole of claim 5, further characterized by an X-ray diffraction pattern peaks at about 5.9, 9.0, 17.7 and 26.1±0.2 degrees two theta.

7. The crystalline solid form of lansoprazole of claim 5, further characterized by an X-ray diffraction pattern substantially as depicted in FIG. 2.

8. The crystalline solid form of lansoprazole of claim 5, further characterized by FTIR absorption b and s at 744, 825, 859, 917, 980, 1023, 1083, 1110, 1260, 1275, 1299, 1311, 1460, 1582, 2810, 2883 and 3014 cm⁻¹.

9. The crystalline solid form of lansoprazole of claim 5, further characterized by a FTIR spectrum substantially as depicted in FIG. 5.

10. A crystalline solid form of lansoprazole, characterized by data selected from the group consisting of an X-ray diffraction pattern having peaks at about 11.4, 14.4, 17.1, 22.9, 28.7 and 34.7±0.2 degrees two theta and a FTIR spectrum having absorption bands at 922, 1040, 1117, 1163, 1266, 1282, 1402, 1456, 2931, 2985 and 3235 cm⁻¹.

11. The crystalline solid form of lansoprazole of claim 10, further characterized by an X-ray diffraction pattern substantially as depicted in FIG. 3.

12. The crystalline solid form of lansoprazole of claim 10, further characterized by FTIR absorption bands at 750, 801, 813, 857, 972, 1087, 1172, 1243, 1254, 1299, 1308, 1443, 1476 and 1581 cm⁻¹.

13. The crystalline solid form of lansoprazole of claim 10, further characterized by a FTIR spectrum substantially as depicted in FIG. 6.

14. A method of preparing crystalline lansoprazole form A, comprising the steps of:
a) preparing a solution of lansoprazole in a solvent selected from the group consisting of methanol, n-butanol, acetone, methylethylketone, ethyl acetate, dimethyl sulfoxide, dimethylformamide and their mixtures optionally with water; and
b) isolating crystalline lansoprazole form A.

15. The method according to claim 14, wherein the lansoprazole used in step (a) is crystalline lansoprazole form A.

16. The method of claim 14, wherein the solvent is selected from the group consisting of methanol, n-butanol, acetone, dimethylsulfoxide, dimethylformamide and their mixtures with water.

17. The method of claim 14, wherein the preparing step is performed by heating the solvent at a temperature higher than ambient temperature.

18. The method of claim 14, wherein the solvent is heated to between about 55° C. and 80° C.

19. The method of claim 14, wherein the isolating step further comprises the steps of:
c) precipitating the lansoprazole; and
d) drying the lansoprazole to yield crystalline lansoprazole form A.

20. The method of claim 19, wherein the precipitating step is performed by cooling the solvent to ambient temperature.

21. A method of preparing the crystalline solid form of lansoprazole of claim 1, comprising the steps of:
a) preparing a solution of lansoprazole in a solvent comprising 2-propanol and water; and
b) isolating the crystalline solid form of lansoprazole of claim 1.

22. The method of claim 21, wherein the lansoprazole used in step (a) is crystalline lansoprazole form A.

23. The method of claim 21, wherein the 2-propanol and water in the solution present in a vol./vol. ratio of about 97.5 to about 2.5.

24. The method of claim 21, wherein the 2-propanol and water in the solution are present in a vol./vol. ratio of about 95 to about 5.

25. The method of claims 23 or 24, wherein the preparing step is performed by heating the solvent at a temperature higher than ambient temperature.

26. The method of claim 25, wherein the solvent is heated to reflux.

27. The method of claim 21, wherein the 2-propanol and water in the solution are present in a vol./vol. ratio of about 80 to about 20.

28. The method of claim 21, wherein the 2-propanol and water in the solution are present in a vol./vol. ratio of about 60 to about 40.

29. The method of claim 27 or 28, wherein the preparing step is performed by heating the solvent at a temperature higher than ambient temperature.

30. The method of claim 29, wherein the solvent is heated to between about 55° C. and 80° C.

31. The method of claim 21, wherein the isolating step further comprises the step of cooling the solution.

32. The method of claim 31, wherein the cooling step is performed by cooling the solvent to ambient temperature.

33. The method of claim 21, wherein the isolating step is performed by filtering under vacuum.

34. A method of preparing the crystalline solid form of lansoprazole of claim 5, comprising the steps of:
a) preparing a solution of lansoprazole in a solvent comprising 2-propanol and water;
b) isolating the lansoprazole; and
c) drying the isolated lansoprazole at a temperature below about 40° C. to yield the crystalline solid form of lansoprazole of claim 5

35. The method of claim 34, wherein the lansoprazole used in step (a) is crystalline lansoprazole form A.

36. The method of claim 34, wherein step (a) is performed by heating the solution to a temperature higher than ambient temperature.

37. The method of claim 34, wherein the solution is heated to reflux temperature.

38. The method of claim 34, wherein the lansoprazole in step (b) is the crystalline solid form of lansoprazole of claim 5.

39. The method of claim 34, wherein the isolating step further comprises the step of cooling the lansoprazole.

40. The method of claim 39, wherein the cooling step is performed by cooling the solution to ambient temperature.

41. The method of claim 34, wherein the drying step is performed under reduced pressure.

42. The method of claim 41, wherein the drying step is performed at ambient temperature.

43. The method of claim 42, wherein the drying step is performed overnight.

44. The method of claim 41, wherein the reduced pressure is about 20 mmHg

45. A method of preparing amorphous lansoprazole, comprising the steps of:
a) preparing a solution of lansoprazole in a solvent comprising 2-propanol and water;
b) isolating the lansoprazole; and
c) drying the isolated lansoprazole at a temperature between about 40° to 50° C. to yield amorphous lansoprazole form.

46. The method of claim 45, wherein the lansoprazole used in step (a) is the crystalline lansoprazole form A.

47. The method of claim 45, wherein the preparing step is performed by heating the solution to a temperature higher than ambient temperature.

48. The method of claim 47, wherein the solution is heated to reflux temperature.

49. The method of claim 45, wherein the isolated lansoprazole in step (b) is the crystalline solid form of lansoprazole of claim 1.

50. The method of claim 45, wherein the isolating step further comprising the step of cooling the lansoprazole.

51. The method of claim 50, wherein the cooling step is performed by cooling the solution to ambient temperature.

52. The method of claim 45, wherein the isolating step is performed by filtering under vacuum.

53. A method of preparing a mixture of crystalline solid form of lansoprazole of claim 1 and form A, comprising the steps of:
a) dissolving or slurrying lansoprazole in a solvent comprising 2-propanol; and
b) isolating mixture of crystalline solid form of lansoprazole of claim 1 and form A.

54. The method of claim 53, wherein the lansoprazole used in step (a) is crystalline lansoprazole form A.

55. The method of claim 53, wherein the slurrying step is performed for about 70 hours.

56. The method of claim 53, wherein the isolating step is performed by filtering under vacuum.

57. The method of claim 53, wherein the mixture contains about 50% wt of the crystalline solid form of lansoprazole of claim 1 and 50% wt crystalline lansoprazole form A.

58. A method of preparing the crystalline solid form of lansoprazole of claim 5, comprising the step of grinding lansoprazole.

59. The method of claim 58, wherein the lansoprazole is crystalline solid form of lansoprazole of claim 1.

60. The method of claim 58, wherein the lansoprazole is ground by a mortar and a pestle.

61. A method of preparing the crystalline solid form of lansoprazole of claim 10, comprising the steps of:
a) preparing a solution of lansoprazole in a solvent comprising methanol;
b) exposing the solution to saturated methanol/water vapor; and
c) isolating the crystalline solid form of lansoprazole of claim 10.

62. The method of claim 61, wherein the lansoprazole used in step (a) is crystalline lansoprazole form A.

63. The method of claim 61, wherein the exposing step is performed at about 25° C.

64. The method of claim 61, wherein the exposing step is performed for about two weeks.

65. A method of preparing the crystalline solid form of lansoprazole of claim 5, comprising the step of drying the crystalline solid form of lansoprazole of claim 1 at ambient temperature under vacuum.

66. The method of method claim 65, wherein the drying step is performed overnight.

67. The crystalline solid form of lansoprazole prepared by the process of claim 21.

68. The crystalline solid form of lansoprazole prepared by the process of claim 34.

69. The crystalline solid form of lansoprazole prepared by the process of claim 53.

70. The crystalline solid form of lansoprazole prepared by the process of claim 61.

71. A pharmaceutical composition comprising an effective amount of at least one crystalline solid form of lansoprazole selected from the group consisting of crystalline solid form of lansoprazole of claims 1, 5, and 10; and a pharmaceutical acceptable excipient.

72. The pharmaceutical composition of claim 71, wherein the crystalline solid form of lansoprazole is the crystalline solid form of lansoprazole of claim 1.

73. The pharmaceutical composition of claim 71, wherein the crystalline solid form of lansoprazole is the crystalline solid form of lansoprazole of claim 5.

74. The pharmaceutical composition of claim 71, wherein the crystalline solid form of lansoprazole is the crystalline solid form of lansoprazole of claim 10.
